(19) United States
(12) Patent Application Publication
(10) Pub. No.: US 2009/0264976 A1
(43) Pub. Date: Oct. 22, 2009

(54) Combination Dilator-Embolic Protection Device

(75) Inventor: Shyam Nagasrinivasa, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: MEDTRONIC VASCULAR, INC., Santa Rosa, CA (US)

(21) Appl. No.: 12/104,647

(22) Filed: Apr. 17, 2008

(51) Int. Cl. A61F 2/06

(52) U.S. Cl. 623/1.11; 606/200

(57) ABSTRACT

A combination dilator-embolic protection device and method for simultaneously dilating a stenotic body vessel while providing protection from embolic debris and constant perfusion through a treatment site is disclosed. The device includes an expandable dilator-filtration component having a first filtration segment, a second filtration segment and an interior volume, wherein the first filtration segment has at least one opening of a first size that permits passage of embolic debris into the interior volume and the second filtration segment has openings of a second size that is smaller than the first size, such that the second filtration segment retains the embolic debris within the interior volume. The dilator-filtration component is expandable into apposition with a stenosis in the body vessel to provide a radial distensible force to dilate the stenotic body vessel while capturing embolic debris within the dilator-filtration component and allowing blood flow through the dilator-filtration component.

### FIELD OF THE INVENTION

The invention relates to a medical device for dilation within a body vessel that provides embolic protection and perfusion during the dilation of the body vessel and/or the dilation of a prosthesis to be positioned within the body vessel.

### BACKGROUND

Human blood vessels often become occluded or completely blocked by plaque, thrombi, deposits, or other substances, which reduce the blood carrying capacity of the vessel. Should the blockage occur at a critical place in the circulatory system, serious and permanent injury, or even death, can occur. To prevent this, some form of medical intervention is usually performed when significant occlusion is detected.

A serious example of vascular occlusion is coronary artery disease, which is a common disorder in developed countries and is the leading cause of death in the United States. Damage to or malfunction of the heart is caused by narrowing or blockage of the coronary arteries that supply blood to the heart. The coronary arteries are first narrowed and may eventually be completely blocked by plaque (atherosclerosis), and the condition may further be complicated by the formation of thrombi (blood clots) on roughened surfaces of, or in eddy currents caused by the plaques. Myocardial infarction can result from coronary atherosclerosis, especially from an occlusive or near-occlusive thrombus overlying or adjacent to the atherosclerotic plaque, leading to ischemia and/or death of portions of the heart muscle. Thrombi and other particulates also can break away from arterial stenoses, and this debris can migrate downstream to cause distal embolization.

Various types of interventional techniques have been developed that facilitate the reduction or removal of the blockage in the blood vessel to allow increased blood flow through the vessel. One technique for treating stenosis or occlusion of a blood vessel is balloon angioplasty. A balloon catheter is inserted into the narrowed or blocked area, and the balloon is inflated to expand the constricted area. In many cases, near normal blood flow is restored. However, the application of balloon angioplasty to certain blood vessels has been limited due to the risk of embolism caused by the dislodgement of the stenotic material, which may then move downstream. For example, angioplasty is not the currently preferred treatment for lesions in the carotid artery because of the possibility of dislodging plaque from the lesion, which may then enter the various arterial vessels of the brain and cause permanent brain damage.

Many techniques exist for preventing the release of thrombotic or embolic particles into the bloodstream during such a procedure. Common among these techniques is introduction of an occlusive device or a filter downstream of the treatment area to capture these embolic particles. The particles may then be removed from the vessel with the withdrawal of the occlusive or filtering device. In another common technique, the particles may be removed by an aspiration catheter prior to the withdrawal of the dilation device. Aspiration catheters have also been found useful in removing thrombus prior to crossing underlying atherosclerotic plaque with guidewires and/or treatment catheters. Such preliminary removal of thrombus makes it easier to cross the stenosis and less likely that thrombo-embolic particles will be released into the bloodstream during the procedure. However, both of these techniques require a dilation device and an additional device for preventing the release of thrombotic or embolic particles into the bloodstream.

Another problem with balloon dilators arises from the fact that the balloon is made from essentially impermeable materials. When such a device is expanded to perform the dilation, blood flow is occluded through the blood vessel in which the balloon dilator is being used. Such an occlusion of blood flow may substantially harm the patient, since portions of the body will not receive blood during the procedure. Thus the length of time a balloon dilator may be used to perform a dilation is limited. Occlusion of blood flow is especially an issue when a dilation procedure is being performed in a portion of the circulatory system where there is a branch in the blood vessels, such as where the arch vessels branch from the thoracic aorta. Improper placement of the balloon dilator in the aorta may cause an unanticipated occlusion in blood flow to a branch of the circulation system. In addition to blocking blood flow, impermeable balloon dilators may cause significant blood pressure upstream of the dilator. The increased blood pressure may cause the balloon dilator, and any prosthesis positioned in the blood vessel that was being dilated such as a stent or stent-graft, to effectively be pushed downstream by the blood and moved out of the desired position. As such, accurate placement of prostheses, such as stents and stent-grafts, may be made more difficult.

Therefore, there remains a need in the art for a dilator that addresses the above-described concerns related to occluding blood flow and causing emboli during a dilation procedure.

### SUMMARY OF THE INVENTION

Embodiments in accordance herewith are directed to a combination dilator-embolic protection device for simultaneously dilating a stenotic body vessel or a tubular prosthesis, providing protection from embolic debris and permitting constant perfusion during the interventional procedure. The device includes an expandable dilator-filtration component having a first filtration segment, a second filtration segment and an interior volume, wherein the first filtration segment has at least one opening of a first size that permits passage of embolic debris into the interior volume and the second filtration segment has openings of a second size that is smaller than the first size, such that the second filtration segment retains the embolic debris within the interior volume. The dilator-filtration component is expandable into apposition with a stenosis or a tubular prosthesis in the body vessel to provide a radial distensible force to dilate the stenotic body vessel or tubular prosthesis while permitting body fluids to perfuse through the treatment area.

In an embodiment, the device includes an elongate shaft component defining a lumen that extends between a proximal end and a distal end thereof and an inner shaft component slidably extending through the lumen of the elongate shaft component and the interior volume of the dilator-filtration component. A distal end of the dilator-filtration component is attached to the inner shaft component proximate a distal end thereof and a proximal end of the dilator-filtration component is attached to the distal end of the elongate shaft portion, such that sliding movement of the inner shaft component relative to the elongate shaft component, or vice versa, reduces the distance between the proximal and distal ends of the dilator-filtration component thereby expanding the component.

### BRIEF DESCRIPTION OF DRAWINGS

The foregoing and other features and advantages of embodiments according to the present invention will be apparent from the following description as illustrated in the accompanying drawings. The accompanying drawings, which are incorporated herein and form a part of the specification, further serve to explain the principles of the embodiments described and to enable a person skilled in the pertinent art to make and use the embodiment. The drawings are not to scale.

FIG. 1 is a schematic side view of a portion of a combination dilator-embolic protection system in a delivery configuration.

FIG. 1A is a cross-sectional view taken along line A-A of FIG. 1.

FIG. 2 is a side view of a distal portion of the dilator-embolic protection device of FIG. 1 positioned within a blood vessel in an expanded configuration.

FIG. 3 is a schematic side view of a combination dilator-embolic protection system.

FIG. 3A is a cross-sectional view taken along line A-A of FIG. 3.

FIG. 4 is a schematic side view of an expandable dilator-filtration component having a stent loaded thereon positioned within a blood vessel.

### DETAILED DESCRIPTION

Specific embodiments are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The following detailed description is merely exemplary in nature and is not intended to be limiting. Although the method and apparatus presented describes treatment in the context of blood vessels such as the aorta, the treatment may also be used in any other body passageways where it is deemed useful.

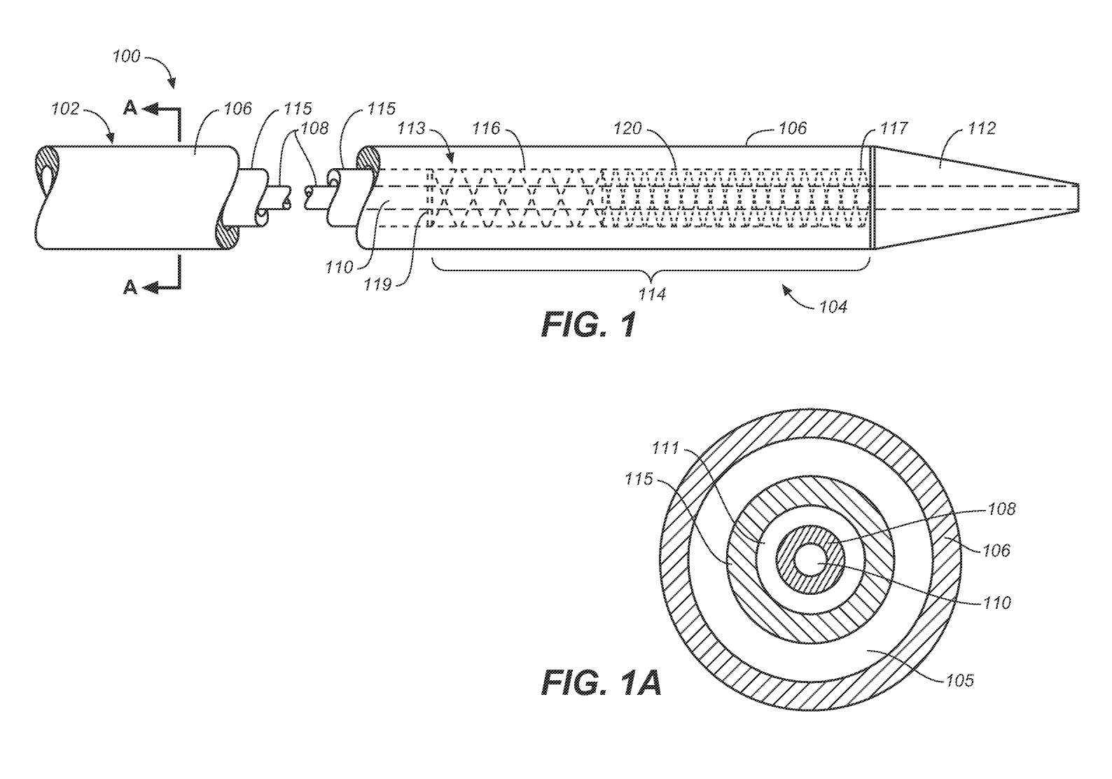

FIG. 1 is a schematic side view of a combination dilator-embolic protection system 100, with FIG. 1A showing a cross-sectional view of the system in FIG. 1 taken along line A-A. System 100 includes a dilator-embolic protection device 113 slidably disposed within a lumen 105 of a sheath catheter 106. An expandable dilator-filtration component 114 of dilator-embolic protection device 113 is positioned within sheath catheter 106 in a distal portion 104 of system 100. An elongate shaft component 115 of dilator-embolic protection device 113 extends proximally from a proximal end 119 of expandable dilator-filtration component 114 within lumen 105 of sheath catheter 106 through a proximal portion 102 of system 100, such that a proximal end (not shown) of shaft component 115 extends out of the patient and may be manipulated by a clinician. A distal end 117 of expandable dilator-filtration component 114 is attached to a distal tip 112 of dilator-embolic protection device 113. The distal tip 112 is formed from a distal end of component 114 and the guidewire shaft 108.

Dilator-embolic protection device 113 also includes an inner shaft component 108 that longitudinally extends the entire length of device 113 through lumen 111 of elongate shaft component 115, an interior volume of expandable dilator-filtration component 114 and distal tip 112. A proximal end (not shown) of inner shaft component 108 extends out of the patient and may be manipulated by a clinician and a distal end of inner shaft component is secured to, or forms a portion of, distal tip 112. Inner shaft component 108 defines a guidewire lumen 110 for receiving a guidewire (not shown) therethrough, such that dilator-embolic protection system 100 may be advanced over an indwelling guidewire to a treatment site within the vasculature. In addition, system 100 may include one or more radiopaque markers (not shown) allowing for accurate positioning of the system within the treatment site.

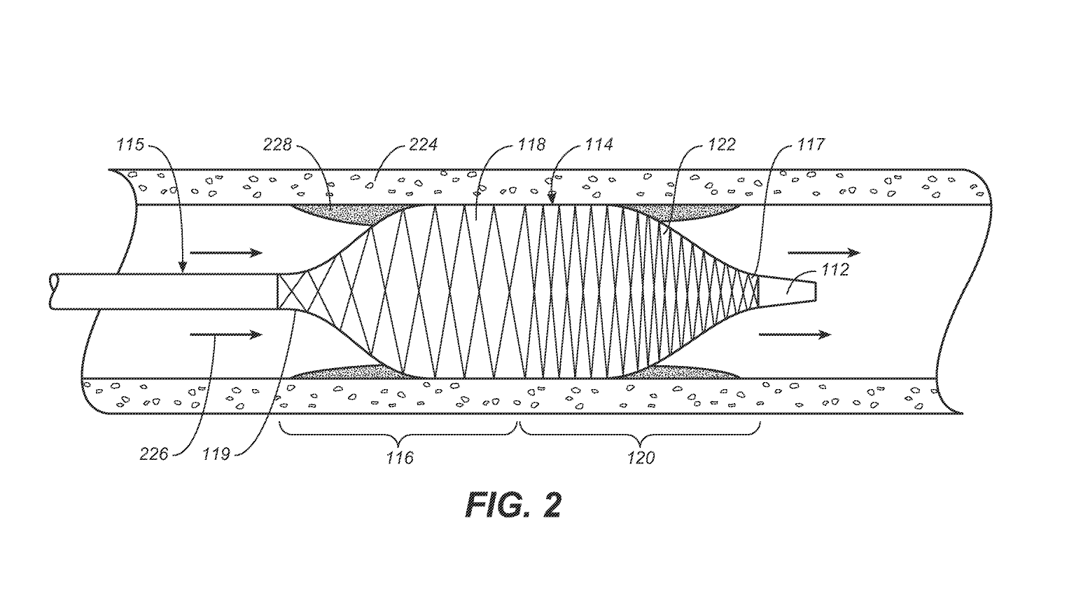

As shown in FIG. 2, expandable dilator-filtration component 114 includes a braided or mesh tubular structure that is expandable into apposition with a stenosis, such as plaque 228, in a body vessel 224 to simultaneously dilate the stenotic vessel and allow perfusion to continue through the treatment site in the direction of arrows 226. Expandable dilator-filtration component 114 includes a first filtration segment 116 defining at least one first opening(s) or gap(s) 118 and a second filtration segment 120 defining a plurality of second openings or gaps 122. First and second openings 118, 122 allow blood or other fluid to flow through expandable dilator-filtration component 114 such that the body vessel is not blocked or occluded during the dilation procedure. In addition to allowing constant perfusion during the dilation procedure, the braided tubular structure of expandable dilator-filtration component 114 also provides protection from plaque particulate, i.e., emboli, that may break-off from the stenosis during the dilation procedure. To provide the filtration function, at least one first opening 118 of first filtration segment 116 is sized to allow passage of plaque particulate dislodged from the stenosis to enter an interior volume of expandable dilator-filtration component 114. To retain or capture the plaque particulate within the interior volume of expandable dilator-filtration component 114, second openings 122 of second filtration segment 120 structure are sized to trap the dislodged plaque particulate and provide embolic protection during the dilation procedure. Thus, second openings 122 of second filtration segment 120 are of a smaller dimension than first opening(s) 118 of first filtration segment 116.

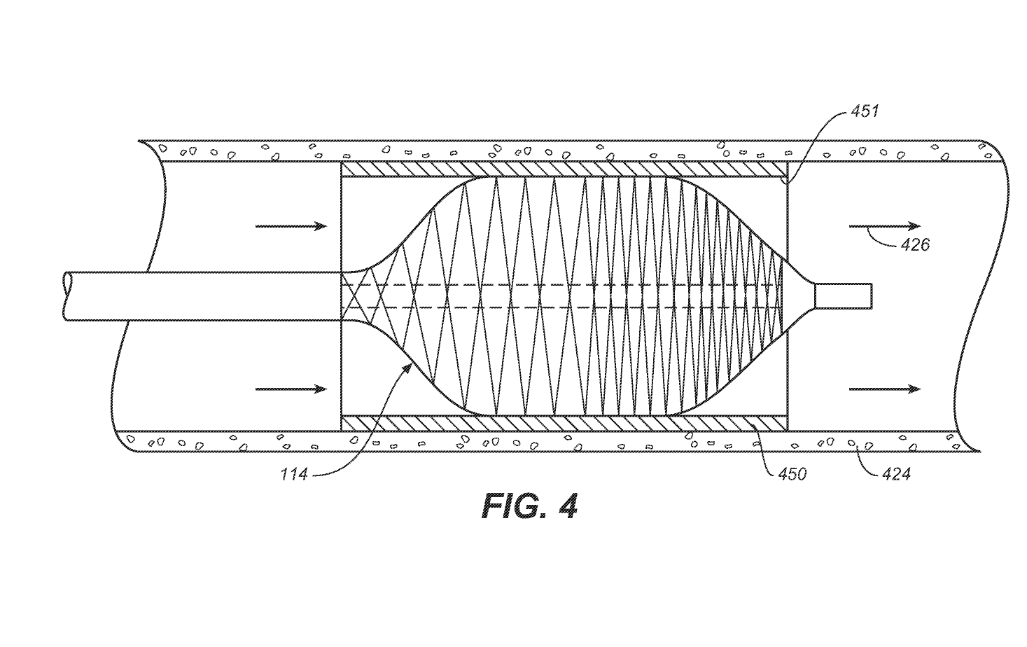

Expandable dilator-filtration component 114 provides a uniform radial distention force to a stenotic body vessel to dilate the body vessel. As discussed with reference to the embodiment of FIG. 4, expandable dilator-filtration component 114 may also be used to deliver a tubular prosthesis to a treatment site within the vasculature and to provide a uniform radial distention force to the tubular prosthesis in order to dilate/deploy the prosthesis in the body vessel. More particularly, expandable dilator-filtration component 114 is transformable between an unexpanded or delivery configuration shown in FIG. 1 to an expanded or deployed configuration shown in FIG. 2. In the delivery configuration, expandable dilator-filtration component 114 is compressed or compacted within sheath catheter 106 to minimize the delivery profile of system 100, which eases advancement of system 100 through the vasculature to the treatment site within body vessel 224. Since the expandable dilator-filtration component 114 may be made of a braided wire or mesh tubular structure that may be crimped into a tighter delivery configuration than a standard angioplasty balloon, a lower French size sheath catheter may be used to deliver dilator-embolic protection device 113 than is customarily used to deliver an angioplasty balloon catheter.

With reference to FIG. 2, sheath catheter 106 is removed at the treatment site and dilator-filtration component 114 is radially expanded into apposition with plaque 228 in body vessel 224 by proximally retracting inner shaft component 110 relative to elongate shaft component 115, which draws proximal and distal ends 119, 117 of expandable dilator-filtration component 114 toward each other. While being expanded, expandable dilator-filtration component 114 provides a balloon-like radially distensible scaffold, such that expandable dilator-filtration component 114 exerts a radial force against plaque 228 to compress plaque 228 against a wall of body vessel 224. When fully expanded within the stenotic body vessel, expandable dilator-filtration component 114 may approximate one of an ellipsoidal, spherical, and cylindrical-like shape.

In FIG. 2, blood is represented as flowing through body vessel 224 and expandable dilator-filtration component 114 in the direction indicated by directional arrows 226 to provide constant perfusion through the treatment area. In the embodiment of FIGS. 1 and 2, first filtration segment 116 is located proximal of second filtration segment 120 such that as blood flows through dilator-filtration component 114, embolic debris may pass through opening(s) 118 of first filtration segment 116 into the interior of component 114 to be trapped therein by second filtration segment 120, which has smaller dimensioned openings 122. In another embodiment where a system in accordance herewith is to be used in a retrograde application, i.e., within a body vessel where blood flows in a direction opposite from that shown in FIG. 2, expandable dilator-filtration component 114 may alternatively be constructed to have a proximal segment with smaller dimensioned openings for retaining embolic debris and a distal segment having one or more openings sized to allow the passage of embolic debris therethrough. In addition the embodiment of FIGS. 1 and 2 include first filtration segment 116 and second filtration segment 120 as each being approximately half of a length of expandable dilator-filtration component 114. In another embodiment, first and second filtration segments 116, 120 may be of unequal lengths.

Expandable dilator-filtration component 114 has sufficient radial strength to dilate a stenotic vessel as it is being expanded. In the embodiment of FIGS. 1 and 2, expandable dilator-filtration component 114 is a braided tubular structure constructed from a plurality of metallic wires or filaments woven together to form first and second filtration segments 116, 120 with openings 118, 122, respectively. Non-exhaustive examples of metallic materials for use in making expandable dilator-filtration component 114 are stainless steel, cobalt based alloys (605L, MP35N), titanium, tantalum, and superelastic nickel-titanium alloy, such as nitinol. In one embodiment, expandable dilator-filtration component 114 may be constructed from a stamped metallic mesh material, wherein the mesh used in first filtration segment 116 would include at least one opening 118 and the mesh used in second filtration segment 120 would include a plurality of smaller dimensioned openings 122. The mesh material is made out of a metallic material with high tensile strength for greater radial distension. Proximal and distal end 119, 117 of expandable dilator-filtration component 114 may be spot welded, laser welded or secured using a bonding sleeve or adhesive to elongate shaft component 115 and inner shaft component 108/distal tip 112, respectively, as would be apparent to one skilled in the relevant art. In another embodiment, at least one of proximal and distal ends 119, 117 of expandable dilator-filtration component 114 may be rotatably connected to elongate shaft component 115 and inner shaft component 108, respectively.

The mesh material is preferably made out of a nickel-cobalt-chromium alloy such as MP35N. The dilator-filtration component 114 can diametrically vary from 20 mm to 40 mm for the aorta for example. The mesh pore size can vary from 50 to 1000 microns which can treat thromboembolic diseases.

Elongate shaft component 115 and inner shaft component 108 may be formed of any suitable flexible polymeric material. Non-exhaustive examples of material for the shaft components are polyethylene terephalate (PET), nylon, polyethylene, PEBAX, or combinations of any of these, either blended or co-extruded. Optionally, a portion of the shaft components may be formed as a composite having a reinforcement material incorporated within a polymeric body to enhance strength, flexibility, and/or toughness. Suitable reinforcement layers include braiding, wire mesh layers, embedded axial wires, embedded helical or circumferential wires, and the like. In an embodiment, the proximal portion of elongate shaft component 115 may in some instances be formed from a metallic tubing, such as a hypotube, or a reinforced polymeric tube as shown and described, for example, in U.S. Pat. No. 5,827,242 to Follmer et al., which is incorporated by reference herein in its entirety. The shaft components may have any suitable working length, for example, 550 mm-600 mm, to extend to a target location within the body vessel.

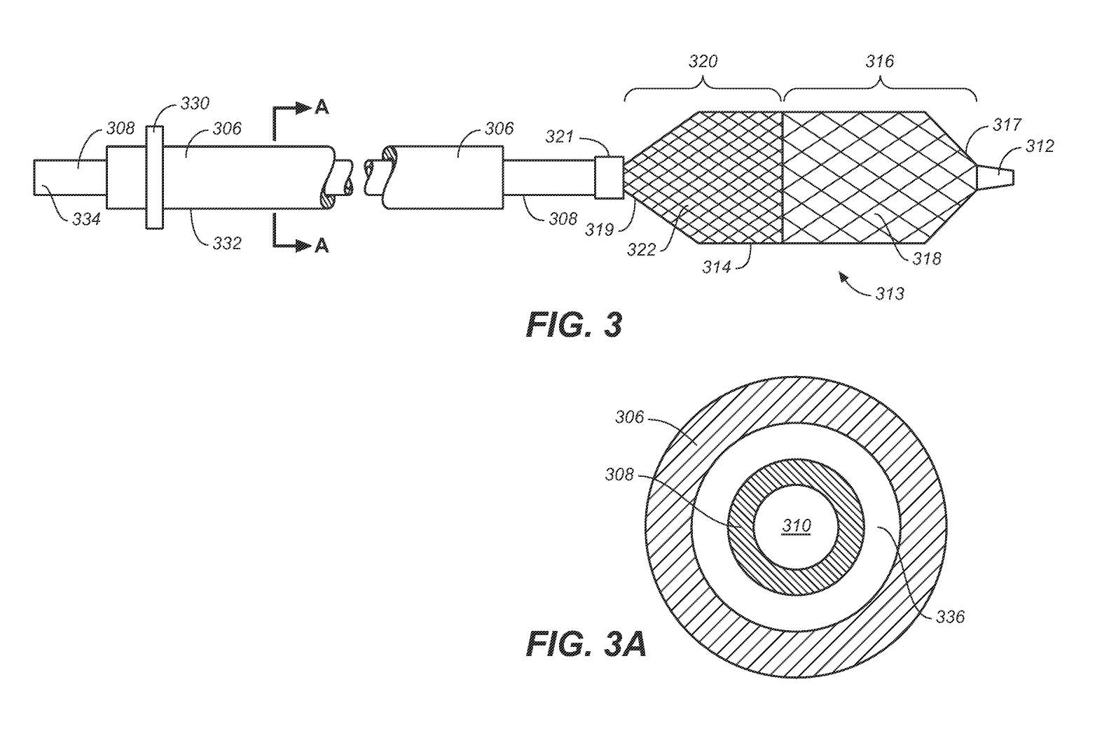

In an embodiment shown in FIG. 3, an expandable dilator-filtration component 314 is self-expanding meaning it has a mechanical memory to return to the expanded, or deployed configuration. Mechanical memory may be imparted to the braided wire or mesh tubular structure that forms expandable dilator-filtration component 314 by thermal treatment to achieve a spring temper in stainless steel, for example, or to set a shape memory in a susceptible metal alloy, such as nitinol. Distal end 317 of expandable dilator-filtration component 314 is fixedly attached by any of the methods noted above to inner shaft component 308 proximate distal end 312 thereof, whereas proximal end 319 of expandable dilator-filtration component 314 is slidably attached to inner shaft component 308 by a hub component 321 to accommodate self-expansion.

In contrast to the previous embodiment, first filtration segment 316 is located distal of second filtration segment 320 such that as a retrograde blood flow passes through dilator-filtration component 314, embolic debris may pass through opening(s) 318 of first filtration segment 316 into the interior of component 314 to be trapped therein by second filtration segment 320, which has smaller dimensioned openings 322.

Expandable dilator-filtration component 314 may be held or biased in its delivery configuration within sheath catheter 306 so that dilator-embolic protection device 313 may be tracked through the vasculature in a low profile. When it is desired to expand dilator-filtration component 314 into the balloon-like radially distensible scaffold configuration, sheath catheter 306 and inner shaft component 308 are moved relative to each other such that expandable dilator-filtration component 314 is released from sheath catheter 306 and allowed to assume its expanded configuration shown in FIG. 3. Inner shaft component 308 may be distally advanced while sheath catheter 306 is held in place or sheath catheter 306 may be proximally retracted while inner shaft component 308 is held in place to cause the relative movement therebetween.

A handle 330 is shown attached to a proximal end 332 of sheath catheter 306 to facilitate securing a longitudinal position or sliding movement thereof relative to inner shaft component 308. In another embodiment, a handle or knob (not shown) may be attached at a proximal end 334 of inner shaft component 308 in order to facilitate securing a longitudinal position or sliding movement thereof relative to sheath catheter 306.

As shown in FIG. 3A, retractable sheath catheter 306 defines a lumen 336 extending therethrough. Inner shaft component 308 slidably extends through lumen 336 of sheath catheter 306. Inner shaft component 308 defines guidewire lumen 310 for receiving a guidewire (not shown) therethrough. Alternatively, the inner shaft component may be a solid rod (not shown) and function as a core wire to provide pushability to the device.

In addition to being utilized for dilating a body vessel, an expandable dilator-filtration component according to an embodiment hereof may be used to expand a tubular prosthesis, such as a stent or stent-graft, within the body vessel. FIG. 4 illustrates a sectional view of stent 450 with expandable dilator-filtration component 114 in apposition with an interior surface 451 of stent 450. Dilator-filtration component 114 exerts a radial distensible force against stent 450 to expand stent 450 until it is fully deployed within body vessel 424. Dilator-filtration component 114 may then be collapsed to its unexpanded configuration and withdrawn from body vessel 424. It will be apparent to one of skill in the relevant art that stent 450 may be crimped on an unexpanded dilator-filtration component 114 for delivery to a treatment site or, in the case of a stent-graft, may be releasably attached thereto by any appropriate attachment means, such as releasable sutures. Similar to previous embodiments, dilator-filtration component 114 permits a clinician to simultaneously dilate stent 450 within body vessel 424 while allowing perfusion to continue through the treatment site in the direction of arrows 426, as well as provides embolic protection during the interventional procedure.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. It will also be understood that each feature of each embodiment discussed herein, and of each reference cited herein, can be used in combination with the features of any other embodiment. All patents and publications discussed herein are incorporated by reference herein in their entirety.

What is claimed is:

1. A combination dilator-embolic protection device [113] for simultaneously dilating a stenosis in a body vessel and providing protection from embolic debris, the device comprising:
an elongate shaft component [115] defining a lumen [111] that extends between a proximal end and a distal end thereof;
an expandable dilator-filtration component [114] having a proximal end [119] attached to the distal end of the elongate shaft portion, the dilator-filtration component having a first filtration segment [116], a second filtration segment [120] and an interior volume, wherein the first filtration segment has at least one opening [118] of a first size that permits passage of embolic debris into the interior volume and the second filtration segment has openings [122] of a second size that is smaller than the first size, such that the second filtration segment retains the embolic debris within the interior volume; and
an inner shaft component [108] slidably extending through the lumen of the elongate shaft component and the interior volume of the dilator-filtration component, the dilator-filtration component having a distal end [117] attached to the inner shaft component proximate a distal end thereof, wherein relative movement between the inner shaft component relative to the elongate shaft component that reduces the distance between the proximal and distal ends of the dilator-filtration component expands the dilator-filtration component into apposition with the stenosis in the body vessel and provides a radial force to dilate the stenosis.

2. The device of claim 1, wherein the first filtration segment is of a first length and the second filtration segment is of a second length that is substantially equal to the first length.

3. The device of claim 1, wherein the first filtration segment is located proximal of the second filtration segment.

4. The device of claim 1, wherein the first filtration segment is located distal of the second filtration segment.

5. The device of claim 1, wherein the dilator-filtration component includes an expandable braided tubular structure that forms the first and second filtration segments.

6. The device of claim 5, wherein the braided tubular structure includes metallic wires.

7. The device of claim 6, wherein the metallic wires are formed from a material selected from the group consisting of a stainless steel alloy and nitinol.

8. The device of claim 1, wherein the dilator-filtration component includes an expandable mesh tubular structure that forms the first and second filtration segments.

9. The device of claim 8, wherein the mesh tubular structure includes a metallic mesh material.

10. The device of claim 1, wherein the first filtration segment includes a plurality of openings of the first size.

11. The system of claim 1, further comprising:
a tubular prosthesis releasably attached to the expandable dilator-filtration component, wherein the dilator-filtration component radially expands and deploys the tubular prosthesis within the body vessel.

12. A method for simultaneously dilating a stenosis in a body vessel while providing protection from embolic debris and continuous perfusion during the interventional procedure, the method comprising:
providing a combination dilator-embolic protection device that includes an expandable dilator-filtration component having a first filtration segment, a second filtration segment and an interior volume, wherein the first filtration segment has at least one opening of a first size that permits passage of embolic debris into the interior volume and the second filtration segment has openings of a second size that is smaller than the first size, such that the second filtration segment retains the embolic debris within the interior volume;
tracking the combination dilator-embolic protection device to the stenosis at the treatment site within the body vessel;
positioning the expandable dilator-filtration component within the stenosis; and
expanding the expandable dilator-filtration component to dilate the stenosis while capturing any embolic debris released from the stenosis within the interior volume of the expandable dilator-filtration component; and
providing continuous perfusion of fluids through the first and second filtration segments of the expandable dilator-filtration component during the interventional procedure.

13. The method of claim 12, wherein the expandable dilator-filtration component includes an expandable braided wire tubular structure that forms the first and second filtration segments.

14. The method of claim 12, further comprising:
providing a tubular prosthesis on the expandable dilator-filtration component; and
concurrently expanding the tubular prosthesis within the stenosis while performing the step of expanding the expandable dilator-filtration component within the stenosis.

15. The method of claim 12, wherein the step of tracking includes performing a retrograde approach to the stenosis and the step of providing a combination dilator-embolic protection device includes the expandable dilator-filtration component having a first filtration segment that is distal of a second filtration segment.

16. The method of claim 12, wherein the step of expanding the expandable dilator-filtration component within the stenosis includes reducing a distance between a proximal end and a distal end of the expandable dilator-filtration component.

17. The method of claim 12, further comprising:
reducing the expandable dilator-filtration component into an unexpanded configuration; and
removing the combination dilator-embolic protection device from the body vessel.
